(19) United States
(12) Patent Application Publication
(10) Pub. No.: US 2014/0276782 A1
(43) Pub. Date: Sep. 18, 2014

(54) CATHETER SYSTEM

(71) Applicant: LARRY D. PASKAR, CHESTERFIELD, MO (US)

(72) Inventor: LARRY D. PASKAR, CHESTERFIELD, MO (US)

(21) Appl. No.: 13/843,823

(22) Filed: Mar. 15, 2013

(51) Int. Cl. A61B 18/14

(52) U.S. Cl. CPC A61B 18/1492
USPC 606/41; 606/130

(57) ABSTRACT

A catheter system that includes a catheter element sized to pass through blood vessels to an organ of an animal. The distal end portion of the catheter element is capable of assuming and maintaining a curved configuration. A medical element is disposed with respect to the catheter element so as to interact therewith, the medical element being capable of assuming and maintaining a curved configuration. At least one ablating or mapping tool is disposed on the distal end portion of either the medical element or the catheter element, or both, for either mapping or ablating tissue in the organ when the distal end portion of the catheter system is disposed in the organ. The catheter system can include a hand grip for controlling the catheter system.

### CROSS-REFERENCE TO RELATED APPLICATIONS

Not Applicable.

### STATEMENT REGARDING FEDERALLY SPONSORED RESEARCH OR DEVELOPMENT

Not Applicable.

### FIELD

The present disclosure relates to a catheter system for use in executing medical procedures within an animal.

### BACKGROUND

This section provides background information related to the present disclosure which is not necessarily prior art.

A catheter is a tubular medical device for insertion into canals, vessels, passageways, or body cavities for diagnostic or therapeutic purposes such as to permit injection/withdrawal of fluids, to keep passageways open, to inspect internal organs and tissues, and to place medical tools into position for medical treatment within the body of an animal.

There is a wide range of medical procedures where a catheter may be used. For example, a catheter may be used to assist the elimination of urine by insertion of a urethral catheter into a patient's body. In other applications, a catheter may be used for clearing of obstructions within a carotid artery or for colonoscopies. A prime example of the use of a catheter includes the treatment of a heart within a human body.

The heart pumps blood as controlled by an electrical conduction system. In that system, an impulse is generated by the sinoatrial node (“SA node”) which is propagated to the fibers of the left and right atria, and the left and right ventricles. The arrival of the impulse initiates coordinated muscle contraction in the four chambers of the heart: the right atrium, the right ventricle, the left atrium and the left ventricle. Coordinated contraction requires that the electrical impulses be distributed to these chambers at the proper time and in the proper sequence. Specifically, there has to be substantial atrial to ventricular delay in the system to allow the atria to completely empty their contents into the ventricles. In part, this is accomplished in a normal heart because the atria are electrically isolated from the ventricles (connected only by means of the atrio-ventricular node (“AV node”)). The AV node briefly delays the electrical impulse in passing from the atria to the ventricles. Timing and synchronization also require that the ventricular cells contract in a coordinated manner. In general ventricular contraction begins at the apex of the heart, progressing upwardly to force blood into the main arteries. The Bundle of His and the Purkinje fibers (collectively, the H-P system) conduct the signal from the AV node to the apex to initiate proper ventricular contraction.

Although normal heart rhythms follow the above-described pathway, abnormal rhythms (arrhythmias) can occur as a result of accessory pathways or dual AV nodal pathways as seen in the Wolfe-Parkinson-White (W-P-W) syndrome, or as a result of electrical abnormalities in the atria or in the ventricles. Examples include atrial tachycardias, atrial flutter, atrial fibrillation, and AV node tachycardias as well as ventricular tachycardias. Many of these arrhythmias can be cured by identifying the abnormal conduction pathway within the cardiac tissue using electrophysiologic studies, i.e., mapping of cardiac activation sequences, and then disrupting or ablating the tissue containing the abnormal tissue through application of energy. The most common energy source to date is radio-frequency energy, although high voltage, direct current, laser, ultrasound, and microwave energy have also been used.

Historically, mapping and ablation were carried out through cardiac surgery which required general anesthesia and cardiopulmonary bypass. The risk was substantial, although the risk was primarily that inherent in the medical procedure and not the mapping and ablation per se. To generally reduce that risk, catheter-based techniques have been developed for mapping and ablation. Such techniques do not require major surgery and, as a result, have significantly decreased side effects and complications.

Patients with Wolfe-Parkinson-White syndrome (W-P-W) in particular are effectively and safely cured by catheter-based ablation techniques. Patients with atrial flutter and focal atrial tachycardias may selectively have their condition controlled by using catheter ablation. The W-P-W syndrome results from an anomalous conduction pathway connecting the atrial muscle directly to the ventricular muscle, thereby short circuiting and bypassing the H-P system. Destruction or ablation of this abnormal pathway can be carried out by delivering energy to the tissue containing the anomalous pathway through an electrode catheter placed in contact with the heart tissue in the left or right heart that can be reached via the peripheral arterial or venous routes commonly used for catheter procedures.

Anomalous conduction pathways can occur between the left atrium and ventricle or between the right atrium and ventricle. Mapping and ablation catheters can be placed in the left heart by one of two approaches. In the first approach, the catheters are introduced into the femoral artery and advanced into the thoracic aorta and through the aortic valve into the left ventricle. The catheter tip is positioned under the mitral valve annulus as close to the abnormal conduction pathway as possible. In the second approach, the catheters are introduced into to the left heart via the left atrium, which is entered through a trans-septal puncture in the fossa ovalis of the interatrial septum from the right atrial side. In this approach, the catheter is introduced into the right atrium through the right femoral vein and the mapping or ablation catheters are positioned on top of (and/or below) the mitral valve annulus as close to the anomalous pathway as possible via the transeptal approach.

Anomalous pathways in the right heart between the right atrium and right ventricle can be approached by one of two venous access routes. The first route targets the pathways ventricular insertion site and is carried out by entering the right atrium from either the inferior or superior vena cava, crossing the tricuspid valve into the right ventricle, and advancing the catheter to the right ventricular apex, causing it to reverse its path backwards so that the catheter tip comes to lie under the tricuspid valve region. The right ventricular insertion under the tricuspid valve is thereby mapped and ablated via this approach.

The second right heart route involves entering the right atrium from the inferior or superior vena cava and locating the atrial insertion site of the anomalous accessory pathway on top of the tricuspid valve annulus on the atrial aspect of the tricuspid valve and then ablating the atrial insertion site.

Successful entry into the heart chamber of interest in no way assures that the tip of a mapping or ablation catheter can precisely be positioned at the focus of an anomalous pathway. It is even less sure that the catheter position can be substantially fixed in a long-term stable position for the ablation procedure, given the cardiac motion as well as the marked variability in chamber size, shape, and internal structural anatomy. Steerable catheters have partially solved this problem of precise positioning and fixation by allowing variable tip curvature radii, but they remain limited in the tip curvatures possible. These steerable catheters do not permit the compound, complex, and out-of-plane shaping capabilities which may be necessary to reach the endocardiac focus by allowing custom curvature of the catheter to conform to the inner contour of the cardiac chamber, thereby permitting wedging of the catheter and subsequent fixation of its tip on the focus of interest.

In past attempts to resolve these issues many different fixed shapes that are disposed at the distal end of a catheter have been used. For example, in catheterization of the left carotid artery of a patient, the end of the catheter can be a very simple shape and still be acceptable for routing the catheter through the patient's body. In a young patient, a simple shape may also be used because the course of the vessels and the shape and size of the vessels and heart are fairly straightforward. As a patient ages, however, the arteries often become dilated and tortuous, resulting in branch vessels coming off at angles different than in the young patient. Likewise, an aged heart may become dilated and deformed, and beat in an irregular rhythm, requiring the catheter to be stabilized against the opposite wall in the heart or a vessel during the routing or treatment procedure. In such circumstances, a single, particular shape might work to route the catheter through the older patient's body, but that specific shape is not determinable until the routing process has already begun. And, if you choose the wrong shape initially when routing the catheter through the older patient, the catheter must be removed and the shape initially selected must be exchanged for a different curved catheter. That process might need to be repeated several times until the selected curve eventually accomplishes the routing of the catheter through that particular area of the patient's body. This can be particularly problematic during heart mapping and ablation procedures and can often take many hours to complete, and in fact, some procedures are ultimately unsuccessful due to the difficulty in routing the catheter precisely to reach and fix upon the focus of aberrant activity. These same routing issues occur during medical treatments that require endoscopes, gastroscopes, laparoscopes, ET tubes, nasogastric tubes, or anywhere a tube must be precisely guided and stabilized from afar.

Other previous inventions use a single pullwire to generate a curve or flatten the distal end of a catheter. However, generating a single curve rarely resolves the complex routing issues that occur during the routing of a catheter through the complex three dimensional tissues and organs of a patient's body. Those types of problematic routing issues require two curves that can be shaped, in situ, and that can be rotationally oriented as needed to pass the catheter system through the patient's body.

Thus, it is clear that a very significant problem exists and that there is a need for a catheter system having a means to shape the distal end of a medical catheter tube in situ, in real time, and under fluoroscopic guidance to modify the shape as necessary to conform to the shape of the vessels or heart chamber, for instance. A shapeable catheter system would obviate exchanges of catheter components while decreasing vessel injury, patient exposure, and other associated morbidity.

### BRIEF SUMMARY OF THE INVENTION

This section provides a general summary of the disclosure, and is not a comprehensive disclosure of its full scope or all of its features.

In accordance with the various embodiments of the present invention, this invention relates to a catheter system having improved placement and fixation capabilities that can reduce the amount of time required in performing medical procedures. More specifically, certain embodiments of the invention provide a catheter system that allows for the interaction of a shapeable distal curve on an inner element of the catheter with a shapeable distal curve on an outer element of the catheter by rotating the inner and outer elements with respect to each other. Some embodiments of the invention include coaxial inner and outer elements that allow a pullwire to shape and generate the curve on the outer element. In those embodiments, the inner element may have a fixed shape that is positioned through activations of the pullwire on the outer element. In alternative embodiments, an additional pullwire may be applied to shape and generate the curve on the inner element. Rotating the distal curves on one or both of the inner and outer elements results in a wide range of shapes to form whole families of down-going, up-going and out-of-plane shapes that can be formed to generate almost any shaped, complex, compound curve in the catheter system. The simplicity of this design allows very complex curves to be formed in smaller catheter systems than would otherwise be needed.

Further areas of applicability will become apparent from the description provided herein. The description and specific examples in this summary are intended for purposes of illustration only and are not intended to limit the scope of the present disclosure.

### DRAWINGS

In the accompanying drawings which form part of this specification:

FIG. 1 is a cross-sectional view of one embodiment of the present invention showing a catheter system being inserted in a heart;

FIG. 2 is a front elevation of the inner medical element of one embodiment of the catheter system showing a possible location of a mapping or ablating electrode;

FIG. 2A is a view similar to FIG. 2, but shown with a cryo-surgery ablation tool;

FIG. 2B is a view similar to FIG. 2, but shown with a microwave ablation tool;

FIG. 2C is a view similar to FIG. 2, but shown with a laser ablation tool;

FIG. 3A-3C are diagrams illustrating exemplary shapes made by moving the medical element translationally with respect to the catheter in which it is located for one embodiment of the present invention;

FIG. 4 is a cross-sectional view of one embodiment of the catheter system illustrating one of a plurality of out-of-plane shapes formable with the present invention;

FIG. 5 is a diagram illustrating that up-going and down-going shapes can be generated by one embodiment of the catheter system by a simple rotation of the medical element with respect to the catheter;

FIG. 6 is a perspective view illustrating indicia which may be used to indicate to the user of one embodiment of the catheter system what type of curve is being generated in the distal end portion the system;

FIG. 7 is a cross-sectional view illustrating forming shapes to map or ablate both above and below a heart valve annulus when using one embodiment of the present invention; and

FIG. 8 is a front elevation of an exemplary hand grip controller for one embodiment of the present invention.

Corresponding reference numerals indicate corresponding parts throughout the several views of the drawings. The drawings described herein are for illustrative purposes only of selected embodiments and not all possible implementations, and are not intended to limit the scope of the present disclosure.

While one embodiment of the present invention is illustrated in the above referenced drawings and in the following description, it is understood that the embodiment shown is merely one example of a single preferred embodiment offered for the purpose of illustration only and that various changes in construction may be resorted to in the course of manufacture in order that the present invention may be utilized to the best advantage according to circumstances which may arise, without in any way departing from the spirit and intention of the present invention, which is to be limited only in accordance with the claims contained herein.

### DETAILED DESCRIPTION OF AT LEAST ONE PREFERRED EMBODIMENT OF THE INVENTION

In the following description, numerous specific details are set forth such as examples of specific components, devices, methods, in order to provide a thorough understanding of embodiments of the present disclosure. It will be apparent to a person of ordinary skill in the art that these specific details need not be employed, and should not be construed to limit the scope of the disclosure. In the development of any actual implementation, numerous implementation-specific decisions must be made to achieve the developer's specific goals, such as compliance with system-related and business-related constraints. Such a development effort might be complex and time consuming, but is nevertheless a routine undertaking of design, fabrication, and manufacture for those of ordinary skill.

In a preferred embodiment of the present invention, the catheter system includes a catheter and a medical element sized to pass through blood vessels to a heart of a patient. It is understood that the patient may be human or non-human. The medical element is generally configured to have an end portion that can be manipulated to assume and maintain various curved configurations that can assist the catheter system as it is routed through the patient, and that allows a medical device attached at the end portion of the medical element to be positioned and operated at the appropriate location within the heart. In alternative embodiments, the medical device may be disposed on one either the catheter or the medical element.

Those of skill in the art understand that routing a catheter through the interior of the body of a patient is a complex task. The complexity of the routing procedure is greatly increased because of the three dimensional shape of the patient's body, internal organs, and vessels. As such, this means that at any point in the catheter routing process, the catheter may need to be routed straight ahead, to the left, to the right, or even reversed upon itself in order to route the catheter through vessels and obstructions found in the patient's body. In fact, if the tip of the catheter is thought of as the inside center point of a sphere, the tip of the catheter may need to be routed in the direction of almost any point on the outer surface of the sphere in order to get the catheter through the vessel or obstruction and to place the tip of the catheter where it is needed. Thus, as the catheter system is routed and used within the patient, certain preferred embodiments of the present invention include the ability to curve the catheter and the medical element into various configurations.

In defining this wide range of curvatures, the disposition of the medical element can be defined in terms of the geometric relationship as it relates to disposition of the catheter. More specifically, it will be understood that in the present embodiments, the catheter system includes a catheter that cooperates operationally with a medical element. The catheter is preferably a substantially straight and cylindrically-shaped tube that is flexible enough to be curved into an arc. When the catheter is curved into a single arc, the axial centerline of the catheter tube is curved to define a first plane. A geometric plane, of course, requires three points to be properly defined, and here, the three points consist of: (1) the point where the axial centerline of the substantially straight catheter tube begins to curve; (2) the point where the curvature of the axial centerline of the substantially straight catheter tube ends: and, (3) any point on the curved axial centerline of the substantially straight catheter tube when the catheter tube is curved.

In a similar manner, the medical element is also generally a substantially straight, cylindrically-shaped, flexible tube that, when curved, defines a second plane. Thus, the separate curves of the catheter tube and the medical element tube result in separately defined planes. It will be appreciated by those skilled in the art that the cooperative association between the first plane of the catheter and the second plane of the medical element is intended to achieve a level of manipulation necessary to allow some preferred embodiments of the catheter system to be routed and positioned within the heart of an animal for execution of the desired medical procedures. In alternative embodiments of the present invention, the catheter system may be adapted for use as an endoscope or a colonoscope. In fact, it will be understood that the various embodiments of the present invention can be readily adapted for any use, and still remain within the intended scope of the claims, where such use requires the insertion of a catheter into the body of an animal and the catheter needs the ability for the catheter system or its components to be shaped into the various curves and configurations as described herein.

Within the various embodiments of the present invention, the most common geometric configurations for the catheter system are: (1) a down-going configuration; (2) an up-going configuration; and, (3) and an out-of-plane configuration. A down-going configuration occurs as shown in FIG. 3A-3C where the catheter system has been placed into a shape in which the tip of the catheter system points in a direction opposed to the direction in which the catheter system would move if it were moved as a unit farther into the patient. An up-going configuration is, of course, the opposite of the down-going configuration and occurs when the catheter system has been placed into a shape in which the tip of the catheter system points in a direction that is the same as the direction in which the catheter system would move if it were moved as a unit farther into the patient.

It will be understood that the up-going and down-going configurations usually exist in a two-dimensional plane. The patient's body, however, is a three dimensional object and the catheter system's configurations need to also be three dimensional. Therefore, the out-of-plane configuration occurs when the first plane, as formed by the curved catheter tube, is not coplanar with the second plane, as formed by the curved medical element tube. An example of an out-of-plane configuration can be shown in FIG. 4.

Turning to the drawings at FIGS. 1-8, a preferred embodiment of a catheter system 11 is shown including a medical element 13 sized to pass through blood vessels to a heart 15 of a patient. The medical element has a distal end portion 17 capable of assuming and maintaining a curved configuration as shown in FIGS. 1-8. A catheter 19 is disposed with respect to the medical element so as to permit manipulation of the medical element 13. The catheter 19 is capable of assuming and maintaining curved configurations such that the distal end portion of the catheter system 11 may be sequentially formed into up-going, down-going, or out-of-plane configurations as needed. In certain embodiments, it is preferred that the distal end portion 21 of the catheter be capable of assuming and maintaining the curved configurations so that the curvable distal end portions 17 and 21 may interact. Depending upon the type of medical procedure to be performed, a medical tool is disposed at the distal end of at least one of either the catheter or the medical element. In certain embodiments of the catheter system, the medical element is disposed within the inside of the catheter in an annular arrangement. In other embodiments, the catheter and the medical element may be in a side-by-side arrangement such that the outer surfaces of the catheter and the medical element are in fixed or non-fixed contact.

In one preferred embodiment, the medical tool is an ablation tool 23 (FIG. 2) that is disposed at the distal end portion of at least one of the medical element 13 and the catheter 19 for ablating tissue in the heart when the distal end portion of the catheter system is disposed in the heart 15. Ablation tools may include cryo-surgery tools (see tool CT in FIG. 2A-note that loop cryo-surgery tools may also be used), microwave tools (microwave antenna MA in FIG. 2B), radiofrequency tools (electrode 23 in FIG. 2), and/or laser tools (tool LA in FIG. 2C), all of which are known in the art. See U.S. Pat. No. 5,733,280 for cryo-surgery ablation tools, U.S. Pat. No. 5,370,678 for microwave ablation tools, and U.S. Pat. No. 6,283,955 for laser ablation tools, the disclosures of which are incorporated herein by reference. Such tools are well-known and these are given for illustrative purposes only.

In the present embodiment, an ablation source may be connected to the ablating tool. The ablation source is normally dependent upon the type of ablation tool used to perform the ablation procedure. For example, an ablation source for cryo-surgery ablation tools is a source of low temperature fluid for passing to the ablation tool, an ablation source for laser ablation tools is a source of laser energy for transmission to the laser ablation tool, an ablation source for a microwave ablation tool is a source of microwave energy for application to the microwave ablation tool, and an ablation source for a radiofrequency ablation tool is a source of radiofrequency power for supplying to the radiofrequency ablation tool.

When an ablation tool is used with the catheter system, the method of ablation includes manipulating an electrophysiology catheter embodiment of the present invention and includes curving an electrophysiology catheter into a desired shape, bending the distal end portion of a catheter disposed in a predetermined relationship with respect to the electrophysiology catheter to fix the electrophysiology catheter in a desired position in a heart, changing the relative positions of the electrophysiology catheter and the catheter either translationally or rotationally to change the position of the electrophysiology catheter in the heart, and ablating or mapping a portion of the heart based upon the position of said electrophysiology catheter in the heart.

In alternative embodiments where mapping of the heart is needed, the ablation tool is replaced with a heart mapping tool. A common mapping tool usually includes an electrode of some type that can locate and identify electrical impulses within the heart. In this embodiment, at least one mapping electrode is disposed at the end portion of at least one of either the catheter or the medical element shaft for mapping electrical signals in the heart when the distal end portion of the catheter system is disposed in said heart. A receiver for accepting communications from the mapping electrode can also be included.

In yet other alternative embodiments, the catheter system uses neither an ablation tool or a mapping tool, but is instead any medical tool that must be routed internally through the body of the patient and the manipulation of the catheter system forms a substantially stable platform in a cavity of the heart from which a medical tool may be extended and used. It is understood that the medical tool used with the catheter system may either be pre-attached to the distal end of at least one of either the catheter or the medical element, or the medical tool can routed through components of the catheter system for placement of the medical tool at the position needed for the selected medical procedure, with the final placement of the medical tool occurring after the catheter system has been placed and positioned in accordance with the descriptions herein.

Medical element 13 (FIG. 3A-3C) is movable translationally (longitudinally) with respect to catheter 19. When the distal end portions of one or both of the medical element 13 and catheter 19 are curved, relative longitudinal movement results in varying the shape of the distal end portion of the catheter system 11, as shown in FIG. 3A-3C. This feature allows the distal end portion of the present embodiment of the catheter system (made up of the distal end portions of both the medical element and the catheter) to form particular shapes as necessary to map or ablate particular tissues in the heart. In FIG. 3A-3C, the shapes shown are all down-going configurations as defined above. Similarly, when medical element 13 (FIG. 4) is moved rotationally with respect to the catheter 19, the medical element forms an out-of-plane configuration, also as defined above. Changing the rotational relationship between the catheter 19 and the medical element 13 can be used to change the shape from down-going to up-going, and vice versa, as shown in FIG. 5.

As shown in FIG. 6, it can be useful to include indicia 31, 33, 35, etc. near the proximal end of the catheter system and near the control mechanism 29 to assist the rotating and translating the medical element with respect to the catheter. Control mechanism 29 can include a first part 29A which is fixed with respect to the catheter 19 and includes a reference indicium 31. The control mechanism 29 can also include a second part 29B which is fixed with respect to the medical element and includes several reference indicia 33, 35, etc. When indicia 31 and 33 are lined up the catheter 19 and medical element 13 curved portions are planar (this is also true when indicium 31 is lined up with indicia (not shown) on part 29B which indicates a 180 degree orientation). The relative position of the indicia indicating anything other than 0 degrees or 180 degrees indicates to the user an out-of-plane configuration of greater or lesser degree. Similar indicia 36A-36D (see FIG. 8), based upon the amount of curvature of the catheter and/or the medical element, can indicate whether the distal end portion of the system is up-going or down-going. It will be appreciated by those skilled in the art that while the indicia shown in the present embodiment are line segments, other types of indicia such as words, numbers, and symbols may also be used while remaining within the intended scope of the present invention. Likewise, it will be understood the certain embodiments of the present invention may also include methods of setting the control mechanism to at least temporarily fix the relationships between the catheter 19 and the medical element 13 as need to brace or maintain the medical tool in a substantially fixed platform or location to position or brace the medical tool when it is performing a medical procedure. When that specific medical procedure is completed, the fixation of the relationship between the catheter 19 and the medical element 13 can be released, and the catheter system can then be repositioned, readjusted, and re-fixated as needed to set up and perform the next medical procedure.

It should be appreciated that these various shapes can be selected and implemented to reduce trauma to the heart during mapping and ablation. Both the medical element 13 and the catheter 19 may be curvable by control elements actuable at the proximal end of the catheter system, or one or both of the medical element and the catheter may have a permanent curvature. Such control elements (like element 29) for controlling pull-wire catheters are known. The system of the present invention is extremely versatile and is capable of mimicking the shape of any commercially available electrophysiology catheter. For example, (see FIG. 7) the catheter system 11 is shapeable into shapes to access the walls of the heart both above and below the tri-cuspid valve. Similarly, the catheter system is shapeable into shapes to access the walls of the heart both above and below the mitral valve, the adjacent the coronary sinus, or any other desired shape.

It should further be appreciated from the above, that shapes can be formed sequentially as needed to map and/or ablate various areas of the heart. Simple translation and/or rotation of the medical element 13 with respect to the catheter 19 result in a vast plurality of desirable shapes.

In an alternative embodiment, the catheter system can include a hand grip device adapted to be held by a human user. The hand grip device cooperates with other components of the catheter system to one of either: (1) adjust the shape of either the catheter 19 or the medical element 13 to result in either an up-going or down-going direction; (2) rotate one of either the catheter or the medical element; or, (3) rotate one of either the catheter or the medical element to modify the planar relationship between the first plane generated by the curve of the catheter and the second plane generated by the curve of the medical element wherein the planar relationship is either coplanar or out of plane. A lever or similar mechanism is provided for rotating the other of the medical element 19 and the catheter tube with respect to the hand grip such that the catheter tube is controllably rotated with respect to the medical element 13. A knob, lever, or the like is also provided for curving the distal end portion of the catheter tube using a pull-wire. (See description below.) Using the hand grip, the catheter system 11 may be controlled to form up-going, down-going and out-of-plane shapes suitable for ablating and/or mapping tissue in a heart.

Turning to FIG. 8, an exemplary hand grip control 137 is shown which is particularly suitable for one-handed control of the catheter system 11. The hand grip control 137 includes a pistol-grip 139 adapted to be held in the hand of a user. A first trigger 141 mounted to the pistol grip 139 is suitably connected to a pullwire 37 or other suitable deflecting device to cause catheter 19 to curve (as desired by the user) to a curved position such as that shown in phantom in FIG. 8. A second trigger 143 mounted to the pistol grip is suitably connected to the medical element 13 to move the medical element translationally (as indicated by the double-headed arrow) with respect to catheter 19 when actuated by the user. Hand grip control 137 also includes a thumb actuable lever 145 to allow the user to rotate catheter 19 with respect to the handle and the medical element 13. When lever 145 is moved by the user in the direction indicated by the arrow in FIG. 8, the catheter 19 is rotated with respect to the medical element 13. Similarly, medical element 13 may be controlled by similar control mechanisms to advance and retract medical element 13 as well as curve the distal end thereof. As noted above, once the components of the catheter system have been routed and properly located, the hand grip control 137 can include elements that can temporarily fix the relationship between the catheter 19 and medical element 13.

The foregoing description of the embodiments of the present invention has been provided for purposes of illustration and description. As various changes could be made in the above constructions without departing from the scope of the invention, it is intended that all matter contained in the above descriptions or shown in the accompanying drawings shall be interpreted as illustrative and not in a limiting sense.

It will also be seen in the above disclosure that several of the intended purposes of the invention are achieved, and other advantageous results obtained. Additionally, in the preceding description, numerous specific details are set forth such as examples of specific components, devices, methods, in order to provide a thorough understanding of embodiments of the present disclosure. It will be apparent to a person of ordinary skill in the art that these specific details need not be employed, and should not be construed to limit the scope of the disclosure.

The disclosure herein is also not intended to be exhaustive or to limit the invention to the precise forms disclosed. Individual elements or features of a particular embodiment are generally not limited to that particular embodiment, but, where applicable, are interchangeable and can be used in a selected embodiment, even if not specifically shown or described.

In the development of any actual implementation, numerous implementation-specific decisions must be made to achieve the developer's specific goals, such as compliance with system-related and business-related constraints. Such a development effort might be complex and time consuming, but is nevertheless a routine undertaking of design, fabrication and manufacture for those of ordinary skill. The scope of the invention should be determined by any appended claims and their legal equivalents, rather than by the examples given.

Terms such as “proximate,” “distal,” “upper,” “lower,” “inner,” “outer,” “inwardly,” “outwardly,” “exterior,” “interior,” and the like when used herein refer to positions of the respective elements as they are shown in the accompanying drawings, and the disclosure is not necessarily limited to such positions. Terms such as “first,” “second,” and other numerical terms when used herein do not imply a sequence or order unless clearly indicated by the context.

When introducing elements or features and the exemplary embodiments, the articles “a,” “an,” “the” and “said” are intended to mean that there are one or more of such elements or features. The terms “comprising,” “including,” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It will be understood that when an element is referred to as being “connected,” “coupled,” “engaged,” or “engageable” to and/or with another element, it can be directly connected, coupled, engaged, engageable to and/or with the other element or intervening elements may be present. In contrast, when an element is referred to as being “directly connected,” “directly coupled,” “directly engaged,” or “directly engageable” to another element, there are no intervening elements present. Other words used to describe the relationship between elements should be interpreted in a like fashion (e.g., “between” versus “directly between,” “adjacent” versus “directly adjacent,” etc.).

What is claimed is:

1. A catheter system comprising:
a medical element sized to be routed through passageways to an organ of a patient, the medical element having a distal end portion for assuming and maintaining a curved configuration;
a catheter element operationally disposed with respect to the medical element so as to permit manipulation of the medical element, wherein the catheter element is capable of assuming and maintaining curved configurations such that the distal end portion of the catheter system can be formed into at least one of either an up-going configuration, a down-going configuration, or an out-of-plane configuration;
at least one medical tool disposed at the distal end portion of at least one of either the catheter element or the medical element wherein the medical tool is used for medical treatment of the organ when the distal end portion of the catheter system is disposed in the patient.

2. The catheter system as set forth in claim 1 wherein the medical element is movable translationally with respect to the catheter element so as to vary the shape of the distal end portion of the catheter system, so as to control the position of the medical tool as required by the shape of the organ.

3. The catheter system as set forth in claim 1 wherein the medical element is movable rotationally with respect to the catheter element to form an out-of-plane configuration to place the medical tool adjacent a portion of the organ inaccessible by any in-plane shape.

4. The catheter system as set forth in claim 1 wherein the medical element is movable rotationally with respect to the catheter element to change the shape of the distal end portion of the catheter system to be at least one of either an up-going configuration or a down-going configuration as needed to position the medical tool into position for treatment of the organ.

5. The catheter system as set forth in claim 1 wherein at least one of either the medical element or the catheter element has an indicia associated therewith to indicate to a user when the medical tool is positioned into one of either an up-going configuration, a down-going configuration, or an out-of-plane configuration, wherein the indicia is disposed near the proximal end of the catheter system.

6. The catheter system as set forth in claim 1 wherein the distal end portion of at least one of either the medical element or the catheter element is curvable by at least one control element actuable at the proximal end of the catheter system.

7. The catheter system as set forth in claim 1 wherein the organ is a heart.

8. The catheter system as set forth in claim 1 wherein the catheter system is shapeable into shapes to access the walls of the heart both above and below a mitral valve of the heart.

9. The catheter system as set forth in claim 1 wherein the catheter system is shapeable into shapes to access the walls of the heart both above and below a tri-cuspid valve of the heart.

10. The catheter system as set forth in claim 1 wherein the medical tool is an ablating tool disposed at the distal end portion of at least one of either the medical element and the catheter element for ablating a tissue in the organ when the distal end portion of the catheter system is disposed in the organ, wherein the ablating tool is selected from the group consisting of cryo-surgery ablation tools, laser ablation tools, microwave ablation tools, and radiofrequency ablation tools, and wherein the selected ablating tool is operatively connected to an ablation source.

11. The catheter system as set forth in claim 1 wherein the medical element is disposed within the catheter element to generate an annular relationship between the medical element and the catheter element.

12. The catheter system as set forth in claim 1 wherein the medical element is disposed adjacent to the catheter element to generate a side by side arrangement between the medical element and the catheter element.

13. The catheter system as set forth in claim 1 wherein the medical element is disposed adjacent to the catheter element to generate a side by side arrangement and wherein the medical element and the catheter element are in fixed contact at their outer surfaces.

14. An electrophysiology catheter system comprising:
a medical element sized to pass through blood vessels to a heart of a patient, wherein the distal end portion of the medical element is capable of assuming and maintaining a curved configuration and defining a plane when in a curved configuration, the distal end portion, when curved, defining a plane;
a catheter element disposed with respect to the medical element for manipulation of the medical element, the catheter element being capable of assuming and maintaining curved configurations such that the distal end portion of the electrophysiology catheter system is formed into at least one of either an up-going configuration, a down-going configuration, or an out-of-plane configuration;
at least one of either an ablation tool or a mapping electrode including a receiver disposed at the distal end portion of at least one of either the catheter element or the medical element for one of either ablating a tissue or mapping signals respectively in the heart when the distal end portion of the electrophysiology catheter system is disposed in the heart.

15. The electrophysiology catheter system as set forth in claim 14 wherein the distal end portion of the medical element is shapeable to be placed adjacent the coronary sinus of the heart.

16. The electrophysiology catheter system as set forth in claim 14 wherein the distal end portion of the electrophysiology catheter system is sequentially shapeable to improve mapping of the heart.

17. The electrophysiology catheter system as set forth in claim 14 wherein the medical element is disposed within the catheter element to generate an annular relationship between the medical element and the catheter element.

18. The electrophysiology catheter system as set forth in claim 14 wherein the medical element is disposed adjacent to the catheter element to generate a side by side relationship between the medical element and the catheter element.

19. A method of manipulating an electrophysiology catheter comprising:
curving an electrophysiology catheter into a desired shape;
bending the distal end portion of a catheter element disposed in a predetermined relationship with respect to the electrophysiology catheter to fix the electrophysiology catheter in a desired position in a heart;
changing a relative position of the electrophysiology catheter and the catheter element one of either translationally or rotationally to change the position of the electrophysiology catheter in the heart; and
ablating or mapping a portion of the heart.

20. The method as set forth in claim 19 wherein the change in the relative position is rotational and the position of the distal end portion of the electrophysiology catheter and the catheter element in the heart is co-planar.

21. The method as set forth in claim 19 wherein the change in the relative position is rotational and the position of the distal end portion of the electrophysiology catheter and the catheter element in the heart is not co-planar.

22. The method as set forth in claim 19 wherein the change in the relative position is translational and the position of the distal end portion of the electrophysiology catheter and the catheter element in the heart is co-planar.

23. The method as set forth in claim 19 wherein the change in the relative position is translational and the position of the distal end portion of the electrophysiology catheter and the catheter element in the heart is not co-planar.

24. The method as set forth in claim 19 further comprising the step of installing the medical tool into the catheter system by routing the medical tool through the inside of the catheter element until the medical tool is disposed at the distal end of one of either the catheter element or the electrophysiology catheter, wherein the catheter element is disposed within the electrophysiology catheter to generate an annular relationship between the catheter element and the electrophysiology catheter.

25. A catheter comprising:
a catheter tube having a first proximal end, a first distal end portion, and a lumen extending therethrough, the catheter tube being suitably sized to be inserted into a human body;
a medical element disposed in a predetermined relationship with respect to the catheter tube, wherein the medical element has a second proximal end and a second distal end portion;
a medical tool that is disposed at the at least one of either the first distal end portion of the catheter tube or the second distal end portion of the medical element;
a hand grip adapted to be held by a human user wherein one of either the medical element or the catheter tube is rotationally fixed to the hand grip so that rotation of the hand grip results in corresponding rotation of at least one of either the medical element or catheter tube respectively;
means for rotating at least one of either the medical element or the catheter tube with respect to the hand grip such that the rotation of the element is controllably rotated with respect to the non-rotating element; and
means for curving at least one of either the distal end portion of the catheter tube or the medical element so that the catheter element is controlled to at least one of either an up-going configuration, a down-going configuration, or an out-of-plane configuration.

26. The catheter as set forth in claim 25 wherein the medical tool is for one of either ablating or mapping a tissue.

27. The catheter as set forth in claim 25 further including means for moving the medical element translationally with respect to the catheter tube.

28. The catheter as set forth in claim 25 wherein the medical element is disposed within the catheter tube to generate an annular relationship between the medical element and the catheter tube.

29. The catheter as set forth in claim 25 wherein the medical element is disposed adjacent to the catheter tube to generate a side by side relationship between the medical element and the catheter tube.

30. A method of manipulating an electrophysiology catheter comprising:
curving an electrophysiology catheter into a shape which conforms to the shape of a cavity in a heart in which the electrophysiology catheter is disposed so as to place at least one of either an ablating tool or a mapping tool at a first desired position in the heart;
moving a distal end portion of the electrophysiology catheter to a second position in the heart; and
recurving the electrophysiology catheter into a second shape that conforms to the shape of the cavity in the heart at the second position within the heart so as to place one of either the ablating tool or the mapping tool at a second desired position in the heart.

31. The method as set forth in claim 30 further comprising the step of installing the medical tool into the catheter system by routing the medical tool through the inside of the catheter element until the medical tool is disposed at the distal end of one of either the catheter element or the electrophysiology catheter, wherein the catheter element is disposed within the electrophysiology catheter to generate an annular relationship between the catheter element and the electrophysiology catheter.

32. An electrophysiology catheter system comprising:
an inner element sized to pass through blood vessels to a heart of a patient wherein the inner element is capable of assuming a curved shape at its distal end portion such that the distal end portion of the inner element, when curved, defines a first plane;
a catheter disposed with respect to the inner element so as to permit manipulation of the electrophysiology catheter system so as to form a stable platform in a cavity of the heart from which a medical element may be extended and used, and wherein the catheter is capable of assuming a curved shape at its distal end portion such that the distal end portion of the catheter, when curved, defines a second plane; and
at least one of either the inner element, the catheter, or the medical element has at least one ablation tool or mapping tool disposed at the distal end portion thereof.
